(19) United States
(12) Patent Application Publication
(10) Pub. No.: US 2019/0247481 A1
(43) Pub. Date: Aug. 15, 2019

(54) AVATAR DENDRITIC CELLS: THE NEOANTIGEN NATURAL KILLER T-CELL CHEMO IMMUNO RADIATION COMPOSITION INDUCING IMMUNOGENIC CELL DEATH

(71) Applicant: NANT HOLDINGS IP, LLC, Culver City, CA (US)

(72) Inventor: Patrick SOON-SHIONG, Culver City, CA (US)

(21) Appl. No.: 16/396,220

(22) Filed: Apr. 26, 2019

Related U.S. Application Data
(63) Continuation-in-part of application No. PCT/US2017/058886, filed on Oct. 27, 2017.
(60) Provisional application No. 62/414,207, filed on Oct. 28, 2016.

(51) Int. Cl. A61K 39/00; A61K 38/20; A61P 35/00

(52) U.S. Cl. CPC A61K 39/0011; A61K 38/2086; A61K 2039/6081; A61K 38/20; A61P 35/00; A61K 38/208

(57) ABSTRACT

Contemplated compositions and methods counteract evasive measures of a tumor by rendering access to the tumor microenvironment, tagging the tumor microenvironment with chemoattractant and/or cytokines, delivering or facilitating a cell-based therapy in the tumor microenvironment while providing inhibition of immune suppressor cells in the tumor microenvironment.

This application is a continuation-in-part of copending International Application PCT/US2017/058,886, with an international filing date of Oct. 27, 2017, which claims the benefit of priority to U.S. provisional application with the Ser. No. 62/414,207, filed Oct. 28, 2016.

### FIELD OF THE INVENTION

The field of the invention is cancer therapy, especially as it relates to cancer therapy with multiple treatment modalities.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Single small-molecule drug cancer treatments generally fail to provide a cure, due to among other things, the high complexity of tumor biology. For the same reason, multi-drug treatment regimes tend to fail in removing all cancer cells from a patient, and relapse is often simply a question of time. More recently, some immune therapy treatments (e.g., checkpoint inhibitor therapy) have reported remarkable success. Unfortunately, while promising, not all of the immune therapy treatments are equally effective and again fail to generate a complete remission.

More recently, it has become apparent that many tumor cells create a complex tumor microenvironment (TME) that typically includes regulatory T cells (Tregs), myeloid derived suppressor cells (MDSCs), and tumor associated macrophages (TAMs) that prevent immune surveillance by endogenous T cells and natural killer (NK) cells, reduce antigen presentation, and hinder the activity of adoptively transferred anti-tumor T cells (Front Surg 2016; 3:11; J Immunol 2008; 181:5425-5432; or Semin Immunol 2016; 28:64-72). Consequently, various attempts have been undertaken to modulate the tumor microenvironment to thereby enhance treatment effects. For example, US 2017/0087185 teaches the use of a lentiviral expression system for the generation of genetically engineered monocytes and monocyte-derived macrophages for immunotherapy. In US 2017/0231995, Bruton's tyrosine kinase (BTK) inhibitors are discussed to interfere with signaling between tumor cells and various immune competent cells within the tumor microenvironment. In yet another approach, as discussed in US 2014/0255341, therapeutic agents are used that increase local production of effector cell-attracting chemokines within a tumor, with concomitant suppression of local production of chemokines that attract regulatory T(reg) cells. For example, such therapeutic agents include Toll-like receptor (TLR) agonists or other activators of NF-KB pathway in combination with a blocker of prostaglandin synthesis or a blocker of prostaglandin signaling, in combination with a type-1 interferon, or in combination with both a blocker of prostaglandin synthesis or signaling and with a type-1 interferon.

While such methods may improve selected aspects of treatment, they still often fail to lead to complete remission of the tumor. Moreover, most of the known treatments may also have systemic effects due to the lack of specificity of action in the tumor microenvironment. Viewed from a different perspective, all or almost all of the known treatments target only a single aspect of tumor biology. Therefore, there remains a need for improved compositions and methods to treat cancer using immune therapy.

### SUMMARY OF THE INVENTION

The inventive subject matter is directed to various compositions and methods where a plurality of treatment modalities are orchestrated in a temporo-spatial manner to condition or reach the tumor microenvironment before immune therapy, and to sustain immune therapy by use of inhibitors of immune suppression. Thus, compositions and methods presented herein represent an multi-stage countermeasure that renders a tumor more susceptible to immune treatment, the attacks the so sensitized tumor by immune therapy, and that sustains immune therapy by reduction of immune suppression. Moreover, contemplated compositions and methods further focus immune therapy to the tumor microenvironment, and most preferably under immune stimulatory conditions.

More particularly, the inventor contemplates treatment methods in which the tumor microenvironment is (preferably first) breached to facilitate tumor cell killing, resulting in tumor necrosis. Proteins associates with tumor necrosis (e.g., nucleolin, histones, etc.) are then used as targets for affinity molecules that also deliver chemokines to the necrotic tissue to so attract various immune competent cells (e.g., native to patient, or recombinant cells) to the tumor microenvironment. In further preferred aspects, immune stimulatory conditions in the tumor microenvironment can be generated using avatar dendritic cells or by using hybrid molecules (e.g, modified nantibodies, TxMs, or Fcabs) that provide at least two distinct portions to attract and/or activate various immune competent cells as described in more detail below. Notably, such hybrid molecules will not directly target cancer cells as treatment agents, but be used to summon various cells of the innate and adaptive arm of the immune system to orchestrate a cell-based immune response against the cancer cell. In addition, the tumor microenvironment may be further treated with one or more compounds that inhibit Tregs, MDSCs, and/or M2 macrophages.

For example, in one aspect of the inventive subject matter, the inventor contemplates method of treating a patient diagnosed with a tumor that includes a step of breaching a vasculature feeding the tumor to thereby increase delivery of at least one of a drug and an immune competent cell into a tumor microenvironment. In another step, one or more cells are killed within the tumor microenvironment, and a targeting agent comprising a signaling component is delivered to the killed cells in the tumor microenvironment. In a further step, a cell-based therapy (using immune competent cells or an avatar dendritic cell), and/or an inhibitor of immune suppressor cells are provided to the tumor microenvironment. Therefore, it should be appreciated that in contrast to heretofore known technologies, contemplated methods will first generate increased access to the tumor microenvironment, typically to kill at least a fraction of tumor cells, leading to a significant proportion of necrotic (as opposed to senescent or apoptotic) cells. Such necrotic tumor cells are then used as an anchor for a targeting molecule that provides chemoattractant signals and/or immunostimulation to the tumor microenvironment. As will be readily appreciated, a so preconditioned tumor will now be significantly more susceptible to immune therapy Immune therapy can then be further enhanced by use of avatar dendritic cells that deliver a stimulatory signal to the tumor microenvironment based on tumor specific antigenic context. Most typically, contemplated treatments will be additionally enhanced by administration of inhibitors of suppressor cells as is further described in more detail below.

For example, the step of breaching the vasculature may include a step of targeting at least one of a gp60 transporter and a neonatal Fc receptor (FcRn). Among other things, targeting the gp60 transporter may be achieved by contacting the gp60 transporter with a drug coupled to an albumin nanoparticle, while targeting the FcRn may be achieved by contacting the FcRn with a drug that is coupled to an Fc portion of an IgG. Suitable drugs for coupling include various cytotoxic drugs, vascular disrupting agents, and/or cytokines. Alternatively, or additionally, the step of breaching the vasculature may also comprise a step of contacting the vasculature with nitric oxide (NO), IL-2, a VEGF receptor inhibitor, and/or a permeability enhancing peptide (PEP), either systemically or locally. It is further contemplated that the step of killing the cells within the tumor microenvironment is performed using at least one of radiation, low-dose chemotherapy, a drug coupled to an albumin nanoparticle, and a drug coupled to an Fc portion of an IgG.

With respect to the targeting agent, it is contemplated that the targeting agent may include an affinity agent that binds to nucleolin, single strand DNA, a histone, or other fragment characteristic of necrotic cells. Preferably, the affinity agent comprises an antibody or fragment thereof, while the signaling component comprises a chemoattractant (and especially a chemokine that attracts a T-cell, an NK cell, a dendritic cell, and/or a macrophage).

While not limiting to the inventive subject matter, the cell-based therapy may comprise a dendritic cell, an activated dendritic cell, a dendritic cell infected with a virus that contains a nucleic acid encoding at least one of a neoepitope, a cancer associated antigen, and a cancer specific antigen, an avatar dendritic cell (chimeric molecule that comprises (a) a fusion protein with an IL15 receptor portion, an Fc portion, and a first affinity portion, and (b) a fusion protein with an IL15 ligand portion, and a second affinity portion), an autologous NK cell, an activated NK cell (aNK), a high-affinity NK cell (haNK), a target activated NK cell, a T-cell, and/or a CAR T-cell. Likewise, the nature of the inhibitor of the immune suppressor cells may vary. However, preferred inhibitors include an inhibitory peptide for a mannose receptor, 5-fluorouracil (5-FU), a phosphodiesterase-5 inhibitor, a COX-2 inhibitor, or cyclophosphamide Where desired, treatment may be further assisted by administering IL-2, IL-15, a IL-15 superagonist and/or IL18 to the patient.

Viewed from a different perspective, the inventor also contemplates a method of treating a patient diagnosed with a tumor that includes a step of administering to a tumor microenvironment a chimeric molecule complex that comprises (a) a fusion protein that has an IL15 receptor portion, an Fc portion, and a first affinity portion, and (b) a fusion protein that has an IL15 ligand portion, and a second affinity portion. Most typically, at least one of the first and second affinity portions will bind to a neoepitope, a tumor specific antigen, or a tumor associated antigen. In a further step, an inhibitor of immune suppressor cells is administered to the tumor microenvironment.

Where desired, such method may further include a step of administering to the patient an autologous NK cell, an activated NK cell (aNK), a high-affinity NK cell (haNK), a target activated NK cell, and/or a T-cell. It is further preferred that the step of administering to the tumor microenvironment is performed across the vasculature of the tumor microenvironment and may further comprise a step of increasing permeability of the vasculature of the tumor microenvironment. In addition, contemplated methods will also include a step of treating the tumor microenvironment with a targeting agent that comprises a signaling component (e.g., chemokine) and an affinity agent that binds to at least one of a nucleolin, DNA, and a histone. In such case, it is also contemplated that the method will further comprise a step of killing cells within the tumor microenvironment.

The inventors also contemplate a method of treating a patient diagnosed with a tumor, comprising: administering to a tumor microenvironment a chimeric molecule complex comprising a fusion protein that has an Fc portion and a preferably bispecific Fab portion having two arms. The Fc portion is adapted to bind to an Fc receptor that is present on various immune competent cells such as macrophages, dendritic cells, or innate NK cells. One arm of the bispecific Fab portion comprises a tumor targeting motif such as scFv or Fab (e.g., engineered to specifically bind a tumor target, or a neo-antigen, or tumor associated antigen, or an epitope) while the other arm is engineered to attract and/or activate further immune competent cells (e.g., engineered to include IL-15 to attract and activate dendritic cells). The method may further comprise a step of administering to the tumor microenvironment an inhibitor of immune suppressor cells. The step of administering to the tumor microenvironment may be performed across the vasculature of the tumor microenvironment (e.g., via FcRn receptor at the neovasculature) and/or comprises a step of increasing the permeability of the vasculature of the tumor microenvironment. Furthermore, the method may comprise a step of treating the tumor microenvironment with a targeting agent comprising a signaling component and an affinity agent that binds to at least one of a nucleolin, DNA, and a histone. In some cases, the signaling component is a chemokine and/or an immune stimulatory cytokine.

Therefore, the inventors also contemplate a method of treating a patient diagnosed with a tumor that includes a step of killing cells within a tumor microenvironment, and delivering a targeting agent to the killed cells in the tumor microenvironment wherein the targeting agent further comprises a signaling component. The signaling component is then used to attract a plurality of immune competent cells, and in yet another step, an inhibitor of immune suppressor cells is administered to the tumor microenvironment.

For example, the step of killing cells within the tumor microenvironment may be performed using at least one of radiation, low-dose chemotherapy, a drug coupled to an albumin nanoparticle, and a drug coupled to an Fc portion of an IgG. As noted above, the targeting agent may comprise an affinity agent that binds to at least one of a nucleolin, DNA, and a histone, and the signaling component may comprise a chemoattractant (e.g., attracting at least one of a T-cell, an NK cell, a dendritic cell, and a macrophage). It is further generally contemplated that the immune competent cells will comprise autologous NK cells, activated NK cells (aNK), high-affinity NK cells (haNK), target activated NK cells, T-cells, T-cells expressing a chimeric antigen receptor, and/or dendritic cells expressing at least one of a neoepitope, a cancer associated antigen, and a cancer specific antigen. Where desired, it is further contemplated that permeability of vasculature feeding the tumor microenvironment may be implemented, for example, by contacting the vasculature with at least one of NO, IL-2, a VEGF receptor inhibitor, and a permeability enhancing peptide (PEP).

Traditional, molecularly uninformed treatment regimens of MTD-based chemotherapy, targeted therapy, monoclonal antibody therapy with high dose radiation impair the immune system thereby generating tolerogenic cell death. This enables the evasion of cancer immunosurveillance and facilitates the selection and escape of resistant, heterogenic clones with resultant metastasis and poor long term outcomes in multiple tumor types. In essence, the traditional regimens and current standards of care may inadvertently exacerbate and perpetuate the Escape phase of tumor immunoediting, supporting the immunosuppressive tumor microenvironment, with poor long term outcomes in patients with cancer.

A paradigm change in cancer care is required in which a modernized treatment is based on the biology of the tumor independent of anatomy, utilizing molecular and immunological insights as to the dynamic state of the cancer in its evolution (elimination, equilibrium, and escape) and specifically tailored to the patient's cancer altered genome, to reinstate the patient to an equilibrium state. The NANT Cancer Vaccine is such an approach.

The immunogenicity of cancer cells results from their antigenicity, (i.e., the expression of MHC restricted specific tumor antigens and tumor neoantigens) and their adjuvanticity, (i.e., the expression or release of damage associated molecular pattern or DAMP).

One particular way to elicit DAMPs within the tumor microenvironment is immunogenic cell death (ICD), a functionally specific type of apoptosis that stimulates tumor-specific immune responses. In turn, low-dose metronomic chemotherapy and low-dose radiation are potent DAMP inducers. The immunogenicity of cell death relies on at least three independent events, namely:
a. The preapoptotic exposure of the endoplasmic reticulum (ER) chaperone protein calreticulin (CRT) and perhaps other chaperones such as HSP70 and HSP90 (17), at the cell surface,
b. The subsequent autophagy-dependent active secretion of adenosine triphosphate (ATP) and;
c. The post apoptotic release of the nuclear nonhistone chromatin-binding protein high mobility group box 1 (HMGB1).

The notion that the tumor tissue itself could act as a source of both antigenicity and adjuvanticity is exploited by the NANT Cancer Vaccine.

The NANT Cancer Vaccine is a modern, regenerative advanced therapeutic approach to cancer, based on these fundamental principles, that an intact innate immune system is necessary to protect against cancer formation during the normal evolutionary process of replication error in physiological stem cell generation. When this system is overwhelmed, the tumor enters into an escape phase resulting in clinical evidence of cancer.

The normal physiological protective immune system of Elimination can be reinstated by the NANT Cancer Vaccine, first by overcoming the immunosuppressed Escape state, followed by induction of immunogenic cell death and activation of effector immune cells, with restoration of the patient to a state of Equilibrium, a paradigm change in cancer care.

Various objects, features, aspects and advantages of the inventive subject matter will become more apparent from the following detailed description of preferred embodiments, along with the accompanying drawing.

### BRIEF DESCRIPTION OF THE DRAWING

FIG. 1 is a schematic exemplary illustration of the three phases of cancer immunoediting, elimination, equilibrium, and escape.

FIG. 2 is a schematic illustration of the escape phase.

FIG. 3 is an exemplary illustration of penetrating the tumor microenvironment and exploiting immunogenic cell death (ICD) to activate the innate and adaptive immune system.

FIG. 4 is an exemplary illustration of chemotherapeutic agents entering the tumor microenvironment.

FIG. 5 is an exemplarily illustration of an approach addressing the three phases of immunoediting.

FIG. 6 is an exemplary illustration of the NANT cancer vaccine key biological elements administered over 14-day cycle.

FIG. 7 is an exemplary illustration of induction of immunogenic cell death and subsequent durable responses.

FIG. 8 is an exemplary illustration of a schematic treatment schedule and effects by the treatment modalities.

FIG. 9 is an exemplary illustration of a treatment molecule according to the inventive subject matter.

### DETAILED DESCRIPTION

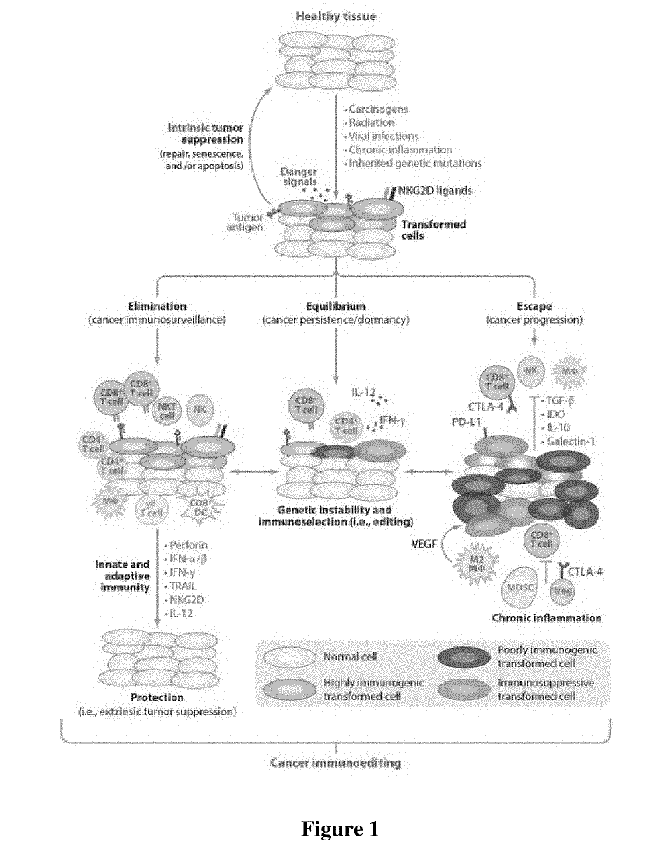

The dynamics of cancer immunoediting by a patient's immune system in its three phases, elimination, equilibrium and escape, provide the foundational basis for both the host-protective mechanisms and tumor evolution of cancer. Understanding these foundational mechanisms of physiological immuno-protection (elimination and equilibrium) and escape associated with cancer formation are the basis of individualized cancer immunotherapies and the development of “The NANT Cancer Vaccine”. FIG. 1 schematically and exemplarily illustrates the three phases of cancer immunoediting, elimination, equilibrium, and escape.

Traditional, molecularly uninformed treatment regimens of maximum tolerated dose (MTD) based chemotherapy, targeted therapy, monoclonal antibody therapy with high dose radiation impair the immune system thereby generating tolerogenic cell death. This enables the evasion of cancer immunosurveillance and facilitates the selection and escape of resistant, heterogenic clones with resultant metastasis and poor long term outcomes in multiple tumor types. In essence, the traditional regimens and current standards of care may inadvertently exacerbate and perpetuate the escape phase of tumor immunoediting, supporting the immunosuppressive tumor microenvironment, with poor long term outcomes in patients with cancer.

The NANT Cancer Vaccine is a modern, regenerative advanced therapeutic approach to cancer, based on these fundamental principles that an intact innate immune system is necessary to protect against cancer formation during the normal evolutionary process of replication error in physiological stem cell generation. When this system is overwhelmed, the tumor enters into an escape phase resulting in clinical evidence of cancer. The inventor now hypothesizes that the normal physiological protective immune system of elimination can be reinstated by the NANT Cancer Vaccine and restore the patient with cancer to an equilibrium state, a paradigm change in cancer care.

The complex biology of mitosis and DNA replication carry the inherent possibility that the replication machinery in regenerative cell replacement is inevitably prone to error, compromising the stability of the genome and resulting in transformed cells, ultimately leading to cancer formation. In the normal state the body is in a phase of Equilibrium under the protection of an intact innate and adaptive immune system. The concept that the innate immune system, which so effectively protects the host from microbial and parasitic pathogens, might also recognize and destroy tumor cells, the Elimination phase, was conceived over a century ago by Paul Ehrlich in 1909. Thus, cancer may arise as a genetic disease by an evolutionary process where somatic cells acquire multiple mutations that overwhelm the protections that normally restrain their uncontrolled expansion, entering into an Escape phase, with clinical evidence of cancer.

The notion that formation of transformed (“cancer”) cells occur routinely as part of the physiological process of regeneration, and that clinical evidence of cancer is kept at bay during this dormancy phase (Equilibrium) by the intact innate immune system of natural killer cells (the Elimination phase), as a normal physiological daily phenomenon in man, is intriguing. When this physiological state is overwhelmed by mutations or by the immunosuppressive state of the tumor microenvironment, the Escape phase ensues, with resultant clinical evidence of cancer. The NANT Cancer Vaccine has been developed based on this notion of the dynamic evolution of cancer, and the capability to restore a state of Equilibrium in a patient with clinical evidence of cancer.

Maximum Tolerated Dose (MTD-Based) Chemotherapy as the Standard of Care and Basis of Drug Development—The Illusion of Clonal Dominance and the Exacerbation of a Tumor Immunosuppressive State: Current standards of care involve administering MTD-based chemotherapy and radiotherapy that significantly impair the patient's immune defenses. This standard practice and the basis of chemotherapy drug development has been propagated for over 40 years on the illusion that cancer resulted from a single mutated clone, growing in a linear fashion. With the toxicities of chemotherapy drug development evolved to targeted therapy, on the basis that single agent targeted therapy will be the answer to the toxicity.

The scientific community has now realized that this long held assumption that cancer cells grow in a linear fashion from a single clonally dominant mutant cell is incorrect. This insight has significant outcome implications both for the practice of high dose chemotherapy, as well as for the administration of single agent targeted therapy. Over the last several years scientists studying the cancer process have elucidated the fact that the vast majority of cancers arise and progress due to numerous mutations in cancer cells, and that cancer is a multi-clonal disease. Moreover, for the most part, each patient's cancer is unique in terms of the nature and number of mutations. It has now been recognized that this is one of the major reasons why many existing therapeutic regimens designed to target a single or even a few mutations have had limited success to date.

Clinical oncologists tend to ignore the significance of the host's intact immune system and have been trained to treat cancer as a cell intrinsic and an anatomy specific phenomenon, with a goal of destroying the tumor cell using MTD based chemotherapy regimens, while overlooking the value of the innate and adaptive immune system to the therapeutic response.

This paradoxical situation exists as it relates to our current standard of care—that traditional MTD-based treatment regimens may be eliciting a short-term response but at the same time driving the patient's equilibrium phase into the escape phase by tilting the balance of the tumor microenvironment into an immunosuppressive state. This insight into the potential cause for limited long-term remissions in most solid tumors following standard of care, requires a paradigm shift in the delivery of MTD-based chemotherapy and single-agent targeted therapy. Traditional, molecularly uninformed treatment regimens of MTD-based chemotherapy, targeted therapy, monoclonal antibody therapy with high dose radiation impair the immune system thereby generating tolerogenic cell death, enables evasion of cancer immunosurveillance and the selection and escape of resistant, heterogenic clones, with resultant metastasis and poor long-term outcomes in multiple tumor types. In essence, the traditional regimens and current standards of care may inadvertently exacerbate and perpetuate the Escape phase of tumor immunoediting, by supporting the immunosuppressive tumor microenvironment resulting in poor long-term outcomes in patients with cancer.

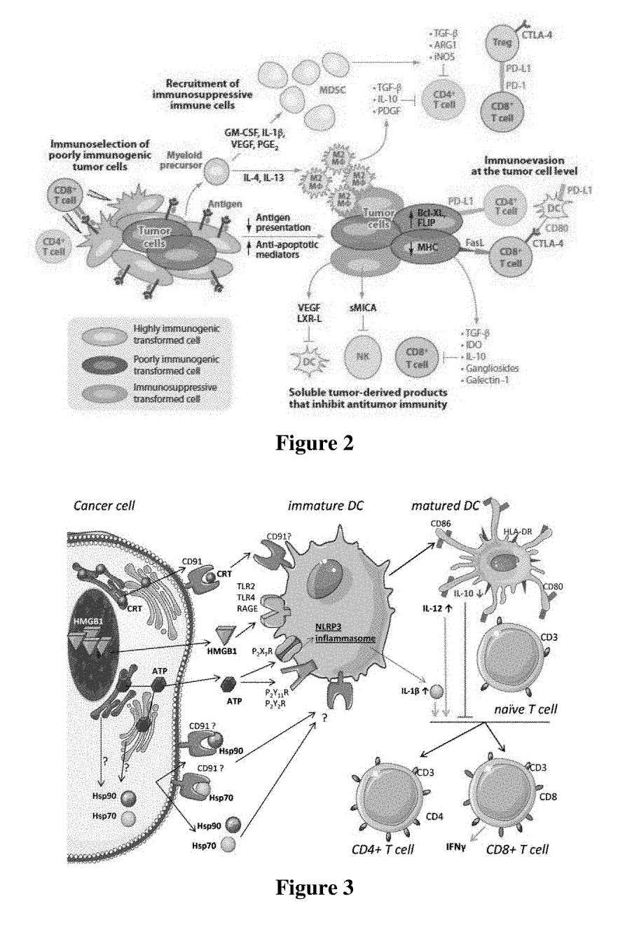

The Immunosuppressive Tumor Microenvironment: Tumor growth represents an outcome of tumor cells escaping host immune surveillance. A major barrier is represented by the presence of immunosuppressive factors that appear to be predominant in cancer patients. These immunosuppressive components include Tregs, myeloid derived suppressor cells (MDSCs), M2 macrophages and immunological checkpoints mediated by cell surface molecules such as CTLA-4 and PD-1. These cells also secrete immunosuppressive cytokines such as TGF-β and IL-10. Studies have shown that these tolerance mechanisms can be induced by tumor and surrounding stromal cells. FIG. 2 provides a schematic illustration of the escape phase.

It should be noted that the escape phase represents the failure of the immune system either to eliminate or to control transformed cells, allowing surviving tumor cell variants to grow in an immunologically unrestricted manner Cancer cells undergoing stochastic genetic and epigenetic changes generate the critical modifications necessary to circumvent both innate and adaptive immunological defenses. Moreover, the immune system contributes to tumor progression by selecting more aggressive tumor variants, suppressing the antitumor immune response, or promoting tumor cell proliferation. The interaction between a heterogeneous population of cancer cells undergoing rapid genetic modifications and the constant immunological pressure exerted by immune cells allows for the Darwinian selection of the most fit tumor variants to survive and form overt cancer in immunocompetent hosts. Thus, nearly all human cancers and experimental cancer cell lines are those that have evaded immunological control.

The NANT Cancer Vaccine is designed to overcome the evasion of immunological control by abrogating the immunosuppressive tumor microenvironment and reversing the Escape phase; to reinstate the innate and adaptive immune system, the Elimination phase, and to restore the Equilibrium dormancy phase. The phase of reversing the immunosuppressive state is accomplished by penetrating the tumor microenvironment to inhibit the tumor immunosuppressed T Reg cell, myeloid derived suppressor cells (MDSCs), M2 macrophages and immunological checkpoints, informed by tissue and liquid biopsies, with low-dose metronomic combination chemotherapeutic agents, peptides and HDAC inhibitors capable of both inducing immunogenic cell death (ICD) with inhibitors of immunosuppressive cytokines.

An exemplary illustration of penetrating the tumor microenvironment and exploiting immunogenic cell death (ICD) to activate the innate and adaptive immune system is shown in FIG. 3. The Elimination Phase—Immunogenic cell death results in the release of soluble mediators occurring in a defined temporal sequence and changes in the composition of the tumor cell surface (DAMP response). For example, the immune system has evolved to recognize and eliminate dying and dead cells and translate cell stress through preapoptotic exposure of calreticulin (CRT) and other endoplasmic reticulum (ER) proteins at the cell surface, secretion of ATP as well as release of the nonhistone chromatin protein high-mobility group box (HMGB1).

The sequential administration of the NANT Cancer Vaccine is to overcome the Escape phase by eliminating the suppressor cells and inducing the Elimination phase by eliciting DAMP response through the use of standard chemotherapy. The scientific community has demonstrated the immunomodulatory effects of metronomic low-dose chemotherapy. This immunomodulatory effect combined with low dose metronomic chemotherapy must be explored as a new paradigm in cancer care to overcome the suppressive tumor microenvironment in the Escape phase of cancer evolution and transition to the Elimination phase.

Accumulating evidence indicates that conventional chemotherapeutic agents, historically thought to act through direct killing of tumor cells may indeed have several off-target effects directed to the host immune system, by inducing immunogenic cell death via the release of DAMP's.

Chemotherapeutic agents may stimulate both the innate and adaptive arms of the immune system by inducing an immunogenic type of cell death in tumor cells resulting in the induction of specific damage associated molecular pattern (DAMP) signals. These signals trigger phagocytosis of cell debris, promoting maturing of dendritic cells, activation of T & NK cells, ultimately resulting in anti-tumor responses. A key element of the scientific rationale of the NANT Cancer Vaccine is exploiting these immunogenic cell death properties of certain chemotherapeutic agents administered in a low-dose metronomic fashion.

An opportunity to reset the immune system in disequilibrium towards activation of a long lasting protective immune response through inducers of immunogenic cell death and DAMP expression is a fundamental scientific basis and rationale for the NANT Cancer Vaccine.

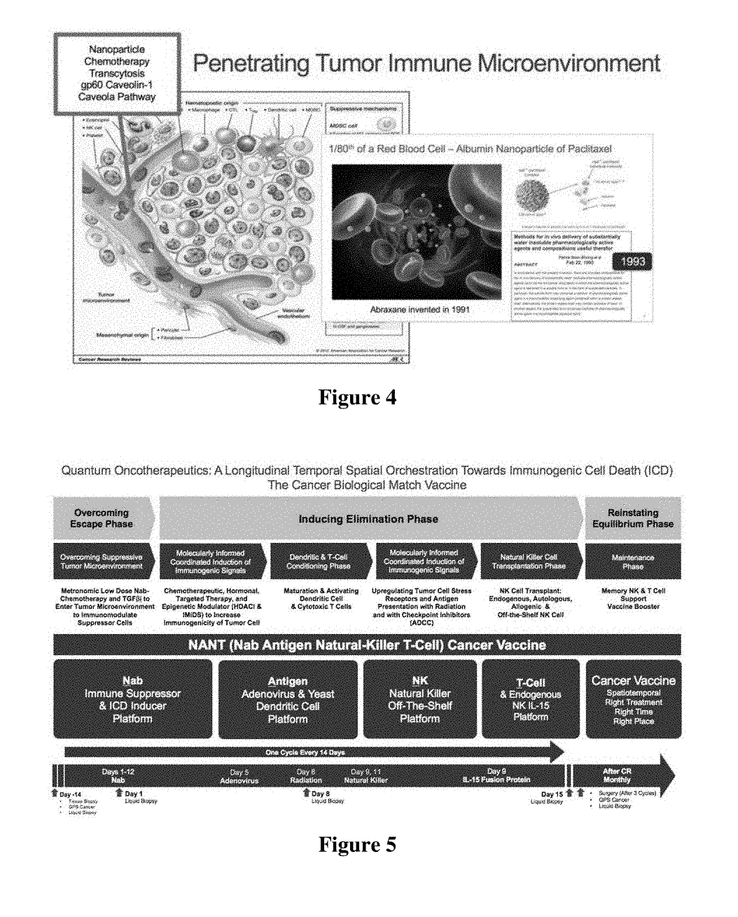

Multiple conventional cytotoxic drugs have demonstrated the capacity to immunomodulate the tumor and induce immunogenic cell death as evidenced by the figure below. Cancer cells undergoing apoptosis while admitting a spatiotemporally defined combination of signals that render them capable of eliciting a long term protective anti-tumor immune response can be exploited through the use of agents such as cyclophosphamide, doxorubicin and Oxaliplatin, cisplatin and paclitaxel. FIG. 4 provides an exemplary illustration of chemotherapeutic agents entering the tumor microenvironment. To enhance localized activity of these agents in the tumor microenvironment, the property of transcytosis via the gp60 Caveolin 1-Caveola pathway is exploited by combining these agents with nanoparticle albumin bound (nab) molecules such as nab-paclitaxel.

The inventive subject matter is directed to compositions and methods that promote, in the context of a tumor microenvironment, activation, proliferation and memory cell formation of NK cells and CD8⁺ T-cells, activation of dendritic cells, and activation of B-cells, while at the same time suppressor cells (e.g., Tregs and myeloid derived suppressor cells (MDSC)) are inhibited. Most preferably, treatment is rendered specific to the tumor microenvironment by targeting necrotic cells in the tumor microenvironment, which serve as an anchor to one or more therapeutic modalities that have binding affinity and specificity to one or more proteins exposed in necrotic cells. Viewed from a different perspective, the treatments contemplated herein will first breach or penetrate the tumor microenvironment and then ‘tag’ the tumor in a location specific manner with a targeting agent that effects signaling to and/or activation of various immune competent cells Immune therapy is then administered to and/or stimulated in the patient, preferably using tumor and patient-specific neoepitopes. Moreover, where desired, immune therapy can be further augmented by administration of immune stimulatory cytokines and/or inhibitors of suppressor cells such as Tregs, MDSC, and M2 macrophages.

To that end, compositions and methods are contemplated that allow/facilitate access to the tumor microenvironment by various drugs and cells, as well as affinity agents that ‘tag’ tumor cells, and most preferably necrotic tumor cells, with one or more chemoattractants that facilitate and/or maintain a cell-based therapy. As should be appreciated, cell-based therapies may rely on endogenous immune competent cells, genetically engineered immune competent cells, and/or avatar dendritic cells as is further discussed in more detail below. An activating tumor microenvironment may further be maintained by exogenous or recombinant cytokines (e.g., IL-15) while ‘tagging’ of the tumor cells may be enhanced by conventional methods, including radiation and chemotherapy.

In contemplated aspects of the inventive subject matter, it should be appreciated that the vasculature feeding the tumor may be breached in various manners, either directly by use of permeability enhancing agents or indirectly via use of molecules that are actively transported across the vascular barrier (e.g., receptor mediated transcytosis or pinocytosis).

For example, access to the tumor microenvironment may be obtained across the epithelial cells using specific receptors present in the neovasculature of the tumor. Most advantageously, such receptors are transport receptors involved in transcytosis and/or pinocytosis. Consequently, preferred receptors for access to the tumor microenvironment include the gp60 receptor and/or the neonatal Fc receptor (FcRn). Therefore, in especially preferred aspects of the inventive subject matter, one or more pharmaceutically active agents can be coupled to albumin or the Fc portion of an antibody. As should be readily appreciated, such coupling may be covalent coupling (e.g., as fusion protein or via a linker) as well as non-covalent coupling (e.g., via hydrophobic interaction of the Sudlow-II domain in albumin) As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously. Among other things, contemplated pharmaceutically active agents include cytotoxic drugs, antimetabolites, tubulin disrupting agents, DNA intercalating agents or DNA alkylating agents, etc. while further contemplated treatment components especially include nanoparticle albumin bound (Nab) chemotherapy combinations.

For example, albumin drug conjugates may be used to exploit the gp60-mediated transcytosis mechanism for albumin in the endothelium of the tumor microvasculature. Thus, various drug conjugates with albumin are contemplated in which a drug is non-covalently coupled to albumin (or nanoparticulate refolded albumin), and contemplated drugs include various cytotoxic drugs, antimetabolic drugs, alkylating agents, microtubulin affecting drugs, topoisomerase inhibitors, drugs that interferes with DNA repair, etc. Therefore, suitable drugs include Bendamustine, Bortezomib, Cabazitaxel, Chlorambucil, Cisplatin, Cyclophosphamide, Dasatinib, Docetaxel, Doxorubicin, Epirubicin, Erlotinib, Etoposide, Everolimus, Gefitinib, Idarubicin, Hydroxyurea, Imatinib, Lapatinib, Melphalan, Mitoxantrone, Nilotinib, Oxiplatin, Paclitaxel, Pazopanib, Pemetrexed, Rapamycin, Romidepsin, Sorafenib, Vemurafenib, Sunitinib, Teniposide, Vinblastine, Vinorelbine, and Vincristine. Such conjugates will advantageously be administered in a low dose and metronomic fashion. Further contemplated drugs for conjugation (or use without conjugation) to albumin include drugs that inhibit suppressor cells in the TME, and especially T-reg cells, myeloid derived suppressor cells, and/or M2 macrophages. For example such drugs include cisplatin, gemcitabine, 5-fluorouracil, cyclophosphamide, doxorubicin, temozolomide, docetaxel, paclitaxel, trabectedin, and RP-182 (see e.g., U.S. Pat. No. 9,492,499).

Preferably, and in at least some aspect of the inventive subject matter, administered pharmaceutically active agents may lead to tumor cell death and so generate necrosis in the microenvironment, which can advantageously be used for tagging as is described in more detail below. Additionally, or alternatively, the pharmaceutically active agent may also inhibit one or more types of suppressor cells, such as MDSCs Tregs, and M2 macrophages.

In addition, antibodies and antibody fragments (e.g., monovalent IgG, F(ab′)₂, etc.) may be coupled to the albumin to thereby provide delivery specificity within the tumor microenvironment, or to provide a desired therapeutic effect (e.g., where the antibody or fragment thereof binds a checkpoint inhibition ligand or receptor).

In another example, the tumor microenvironment may be accessed by various antibody-drug conjugates where entry of the antibody-drug conjugate into the tumor microenvironment is mediated by the FcRn receptor of the endothelium of the tumor microvasculature. It should be recognized that antibodies can cross the endothelium of the tumor microvasculature via FcRn-mediated pinocytosis. Therefore, various immunoglobulin conjugates and chimeric proteins (e.g., with the Fc portion of an immunoglobulin) are contemplated. Of course, it should be appreciated that where the tumor microenvironment is accessed by an antibody-drug conjugate, the antibody will have a binding specificity that is specific to a tumor epitope (e.g., tumor and patient specific neoepitope, tumor associated antigen, tumor specific antigen). Such specificity advantageously delivers the drug directly to the tumor cells in the tumor microenvironment.

With respect to suitable drugs, the same considerations as discussed above apply, and particularly preferred drugs include various cytotoxic drugs, antimetabolic drugs, alkylating agents, microtubulin affecting drugs, topoisomerase inhibitors, drugs that interferes with DNA repair, etc. Therefore, suitable drugs include Bendamustine, Bortezomib, Cabazitaxel, Chlorambucil, Cisplatin, Cyclophosphamide, Dasatinib, Docetaxel, Doxorubicin, Epirubicin, Erlotinib, Etoposide, Everolimus, Gefitinib, Idarubicin, Hydroxyurea, Imatinib, Lapatinib, Melphalan, Mitoxantrone, Nilotinib, Oxiplatin, Paclitaxel, Pazopanib, Pemetrexed, Rapamycin, Romidepsin, Sorafenib, Vemurafenib, Sunitinib, Teniposide, Vinblastine, Vinorelbine, and Vincristine, cisplatin, gemcitabine, 5-fluorouracil, cyclophosphamide, (al)doxorubicin, temozolomide, docetaxel, paclitaxel, trabectedin, and RP-182.

Moreover, where the drug is a protein or polypeptide, particularly preferred conjugates and chimeric proteins will include immune stimulatory cytokines (e.g., IL-2, IL15, etc.) and chemokines (e.g., CXCL14, CD40L, CCL2, CCL1, CCL22, CCL17, CXCR3, CXCL9, CXCL10, and CXCL11, etc.). Other suitable proteins that can be coupled to the antibody include various enzymes, such as urease to site-specifically increase pH of the tumor microenvironment, or various proteases to degrade excess collagen.

Therefore, it should be appreciated that access to the tumor microenvironment as discussed herein will advantageously allow preconditioning of the tumor to subsequent treatment, and most typically to immune therapy. Viewed from a different perspective, breaching the tumor microenvironment may be used to reduce immune suppression, to increase the local pH, and/or to generate immune stimulatory conditions.

In still further contemplated aspects, access to the tumor microenvironment may also be obtained by directly or indirectly disrupting the vascular barrier. For example, disruption of the vascular barrier can be achieved by administration of IL-2, a permeability enhancing peptide portion (PEP) of IL-2, bradykinin, NO, arginine, a prostaglandin (especially prostaglandin E2), or a VEGF receptor inhibitor (e.g., bevacizumab), typically in a systemic manner. On the other hand, disruption of the vascular barrier can also be achieved by local administration of NO or a NO precursor or the PEP of IL-2, for example, via a drug eluting stent.

Regardless of the manner of accessing the tumor microenvironment, it should therefore be appreciated that treatment can be provided in a relatively localized and concentrated fashion to so specifically generate treatment conditions suitable to enhance an immune reaction in the tumor microenvironment. In particular and as also described in more detail below, various immune competent cells, avatar dendritic cells, and protein based molecules can be delivered to the tumor microenvironment for focused and localized treatment. Preferably, but not necessarily, permeability enhancers are preferably provided together with or prior to administration of drugs that bind to necrotic tumor cells and/or drugs that inhibit suppressor cells.

With respect to the tumor cell killing it is generally preferred that the cells are exposed to one or more agents and/or conditions that preferably or primarily lead to necrosis or necrotic cell death. Notably, and contrary to many other treatment protocols, tumor cell killing at this stage of treatment is not intended to eradicate all tumor cells but intended to generate tumor cell necrosis in some cells and upregulation of stress signals in other cells. Therefore, it should be appreciated that contemplated treatments will be administered to the patient in a dosage and/or schedule that is not effective to eradicate the entire tumor, or no more than 90% of the tumor, or no more than 80% of the tumor, or no more than 70% of the tumor, or no more than 50% of the tumor. Instead treatments according to the inventive subject matter will produce tumor necrosis in a portion of the treated cells and increased expression of stress signals in another portion of the treated cells to so increase immunogenicity of the tumor.

For example the stress signals produced by radiation and/or chemotherapy will typically include up-regulated expression of damaged associated molecular patterns (DAMP) signals, and up-regulated tumor associated MHC restricted antigens and stress receptor ligands (NKG2D-L) through low-dose radiation and/or low dose chemotherapy.

Tumor cell killing is preferably performed at low dose, preferably in metronomic fashion to trigger overexpression or transcription of stress signals. For example, it is generally preferred that such treatment will be effective to affect at least one of protein expression, cell division, and cell cycle, preferably to induce apoptosis or at least to induce or increase the expression of stress-related genes (and especially NKG2D ligands, DAMPsignals). In this context it should be noted that chemotherapeutic agents may advantageously stimulate both the innate and adaptive arms of the immune system by inducing an immunogenic type of cell death in tumor cells resulting in the induction of specific damage associated molecular pattern (DAMP) signals. These signals trigger phagocytosis of cell debris, promoting maturing of dendritic cells, activation of T- and NK cells, ultimately promoting anti-tumor responses. To take particular advantage of expression and display or secretion of the stress signals, it is generally preferred that low dose chemotherapy and/or low dose radiation is followed within 12-36 by transfusion of NK cells (e.g., aNK cells, haNK cells, or taNK cells) to enhance an innate immune response.

For example, in some contemplated aspects an increase in necrosis and immunogenicity and/or a decrease immune suppression in the tumor microenvironment will include a low dose treatment using one or more of chemotherapeutic agents that target the tumor microenvironment. Most typically, the low-dose treatments will be at dosages that are equal or less than 70%, equal or less than 50%, equal or less than 40%, equal or less than 30%, equal or less than 20%, equal or less than 10%, or equal or less than 5% of the LD₅₀ or IC₅₀ for the chemotherapeutic agent. Viewed from a different perspective, low dose administration will be at dosages of the drug that are between 5-10%, or between 10-20%, or between 20-30%, or between 30-50%, or between 50-70% of a normally recommended dosage as indicated in the prescribing information for the drug. Additionally, where desired, such low-dose regimen may be performed in a metronomic manner as described, for example, in U.S. Pat. Nos. 7,758,891, 7,771,751, 7,780,984, 7,981,445, and 8,034,375.

In addition, contemplated treatments to target the tumor microenvironment to increase necrosis and/or immunogenicity may be accompanied by radiation therapy, and especially low dose targeted stereotactic radiation therapy (e.g., dosages that are between 5-10%, or between 10-20%, or between 20-30%, or between 30-50%, or between 50-70% of normal recommended dosages for radiation of the tumor).

As noted before, tumor cell killing may be performed using chemotherapy and/or radiation in conventional manners, or more preferably in a low dose (metronomic) manner, but may also be combined with the breach of the tumor microenvironment. Therefore, the administration of tumor cell killing drugs may be assisted by coupling the drugs to albumin or antibodies to so take advantage of gp60-mediated or FcRn-mediated transport into the tumor microenvironment.

With respect to suitable targeting agents that are delivered to the killed cells in the tumor microenvironment it is generally preferred that the targeting agent specifically binds to one or more components of a necrotic cell and further comprises a signaling component that provides a signal for immune stimulation and/or acts as a chemoattractant for immune competent cells into the tumor microenvironment. Most preferably, the targeting agent allows for a location specific delivery of the immune stimulation or chemoattractant and targeting is based on various features common to tumor necrosis, which exposes the cell and nuclear skeleton and various nuclear components. Therefore, it is contemplated that the targeting agents will have binding affinity and specificity (e.g., affinity to target of equal or less than 10⁻⁷ M) to nucleolin, single stranded DNA (e.g., forming G-rich quadruplexes), and one or more histone proteins. Consequently, especially preferred agents include antibodies or fragments thereof, which will be coupled to the signaling component. There are numerous antibodies known in the art that target/bind known necrosis related proteins and nucleic acids, and all of those are deemed suitable for use herein.

In further contemplated aspects, it should be recognized that the signaling component may be a chemoattractant, and especially a chemokine that attracts at least one of a T-cell, an NK cell, a dendritic cell, and a macrophage. Therefore, especially suitable chemoattractants include chemokines, and particularly pro-inflammatory chemokines, including CCL2, CCL3, CCL4, CCL5, and CCL11, and CXCL1, CXCL2, CXCL8, and CXCL10. Likewise, it is contemplated that the signaling component may also be an immune stimulatory cytokine, and particularly preferred immune stimulatory cytokines include IL-2, IL-15, a modified IL-15, and IL-21. In addition, it should be appreciated that further immune stimulatory compounds may be provided to the patient, and particularly preferred immune stimulatory cytokines include IL-2, IL15, IL-21, and IL-15 superagonists (and especially ALT-803, an IL-15-based immunostimulatory protein complex comprising two protein subunits of a human IL-15 variant associated with high affinity to a dimeric human IL-15 receptor a).

Regardless of the particular type of signaling component, it is contemplated that the signaling component may be covalently or non-covalently coupled to the targeting agent. For example, covalent coupling may be achieved by formation of a chimeric molecule in which the targeting agent (e.g., antibody) and the signaling component are coupled to each other via a flexible or rigid peptide linker (e.g., having between 5 and 50 amino acids). On the other hand, the targeting agent and the signaling component may also be coupled to each other via a cross-linker that uses thiol or amino groups of the targeting agent and the signaling component. On the other hand, the targeting agent and the signaling component may be non-covalently coupled to each other using hydrogen bonding or hydrophobic interactions, or use mediator molecules that facilitate coupled such as avidin/biotin coupling (where the targeting agent is carries an avidin portion and where the signaling component is biotinylated.

For example, especially suitable targeting agents include anti-nucleolin antibodies or anti ssDNA antibodies or antibodies against DNA/histone H1 complexes (all commercially available as mono and/or polyclonal antibodies), all of which may be modified by a signaling component using conventional crosslinking chemistry. For example, where the signaling component is a chemokine or a cytokine, crosslinking the two proteins may be achieved via bis(sulfosuccinimidyl)suberate. Of course, there are numerous alternative crosslinkers known in the art and all homobifunctional (reactive groups are NHS esters, imido esters, etc.) and heterobifunctional (reactive groups are NHS ester/maleimide, NHS esters/haloacetyl, etc.) crosslinkers are deemed appropriate for use herein. In further contemplated aspects, suitable crosslinkers may also be pH sensitive and include linking moieties such as a (6-maleimido-caproyl) hydrazone.

Upon tagging of the necrotic cells with the targeting agent/signaling component, a cell-based therapy using immune competent cells and/or avatar dendritic cell may be administered to the patient. Of course, it should be appreciated that the cell-based treatment may also recruit the patient's own immune competent cells, especially where the patient's immune system is not suppressed from prior chemotherapy. In addition, or alternatively, autologous cells from the patient may be used that may or may not be genetically modified.

For example, in one aspect of contemplated methods, the immune competent cells are dendritic cells that are genetically modified to express and present via MHC-I and/or MHC-II one or more tumor associates antigens, tumor specific antigens and/or tumor and patient specific neoepitopes (and optionally one or more cytokines and/or co-stimulatory molecules). Of course, it should be appreciated that the dendritic cells may the patient's dendritic cells that were previously infected by a viral vaccine to express these antigens. Alternatively, it is contemplated that the dendritic cells may not express recombinant antigens but be patient naïve cells that migrate to the tumor microenvironment and there take up and present cancer specific antigens (including neoepitopes). Advantageously, and particularly where IL-2 and/or IL-15 was previously administered, the dendritic cells will be in an activated state and thus be effective in activating T-cells towards CD8+ and CD4+ T-cells.

In another aspect of contemplated methods, the immune competent cells may also be NK cells (autologous, or modified heterologous) that migrate towards the tumor microenvironment and upon binding the antibody and/or recognizing NKG2D ligands of cancer and necrotic cells exert direct cytotoxic activity in the tumor microenvironment. The cytotoxic activity then results in a release of more tumor cell proteins, which in turn will generate a further immune response. Moreover, where the immune competent cells are NK92 derivatives, it is generally preferred that these cells are high affinity CD16 NK92 cells (haNKs) or target activated NK92 cells (taNKs) that express a chimeric antigen receptor targeting one or more neoepitopes of the patient's tumor as described in more detail below.

Therefore, it is contemplated that contemplated treatments and uses may also include transfusion of autologous or heterologous NK cells to a patient, and particularly NK cells that are genetically modified to exhibit less inhibition. For example, the genetically modified NK cell may be a NK92 derivative that is modified to have a reduced or abolished expression of at least one killer cell immunoglobulin-like receptor (KIR), which will render such cells constitutively activated. Of course, it should be noted that one or more KIRs may be deleted or that their expression may be suppressed (e.g., via miRNA, siRNA, etc.), including KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and KIR3DS1. Such modified cells may be prepared using protocols well known in the art, or may also be commercially obtained from NantKwest as aNK cells (‘activated natural killer cells). In addition, contemplated NK cells suitable for use herein also include those that have abolished or silenced expression of NKG2A, which is an activating signal to Tregs and MDSCs.

Alternatively, the genetically engineered NK cell may also be an NK92 derivative that is modified to express a high-affinity Fcγ receptor (CD16-158V). Sequences for high-affinity variants of the Fcγ receptor are well known in the art, and all manners of generating and expression are deemed suitable for use herein. Expression of such receptor is believed to allow specific targeting of tumor cells using antibodies produced by the patient in response to the treatment contemplated herein, or supplied as therapeutic antibodies, where those antibodies are specific to a patient's tumor cells (e.g., neoepitopes), a particular tumor type (e.g., HER2, PSA, PSMA, etc.), or antigens associated with cancer (e.g., CEA-CAM). Advantageously, such cells may be commercially obtained from NantKwest as haNK cells (‘high-affinity natural killer cells) and may then be further modified (e.g., to express co-stimulatory molecules or to have abolished or silenced expression of NKG2A).

In further aspects, genetically engineered NK cells may also be genetically engineered to express a chimeric T cell receptor. In especially preferred aspects, the chimeric T cell receptor will have an scFv portion or other ectodomain with binding specificity against a tumor associated antigen, a tumor specific antigen, and/or a neoepitope of the patient as determined by suitable omics analysis. As before, such cells may be commercially obtained from NantKwest as taNK cells (‘target-activated natural killer cells’) and further modified as desired. Where the cells have a chimeric T cell receptor engineered to have affinity towards a cancer associated antigen or neoepitope, it is contemplated that all known cancer associated antigens and neoepitopes are considered appropriate for use. For example, tumor associated antigens include CEA, MUC-1, CYPB1, PSA, Her-2, PSA, brachyury, etc.

Similarly, the immune competent cells may also be cytotoxic T-cells that are either native and attracted by the chemoattractant, or genetically engineered T cells expressing a chimeric antigen or T-cell receptor that binds to a neoepitope of the patient's tumor. Moreover, it should be noted that the methods and uses contemplated herein also include cell based treatments with cells other than (or in addition to) NK cells. For example, suitable cell based treatments include T cell based treatments. Among other options, it is contemplated that one or more features associated with T cells (e.g., CD4+ T cells, CD8+ T cells, etc.) can be detected. More specifically, contemplated omics analysis can identify specific neoepitopes (e.g., 8-mers to 12-mers for MHC I, 12-mers to 25-mers for MHC II, etc.) that can be used for the identification of neoepitope reactive T cells bearing a specific T cell receptor against the neoepitopes/MHC protein complexes. Thus, the method can include harvesting the neoepitope reactive T cells. The harvested T cells can be grown or expanded (or reactivated where exhausted) ex vivo in preparation for reintroduction to the patient. Alternatively, the T cell receptor genes in the harvested T cells can be isolated and transferred into viruses, or other adoptive cell therapies systems (e.g., CAR-T, CAR-TANK, etc.). Beyond neoepitopes, the omics analyses can also provide one or more tumor associated antigens (TAAs). Therefore, one can also harvest T cells that have receptors that are sensitive to the TAAs identified from these analyses. These cells can be grown or cultured ex vivo and used in a similar therapeutic manner as discussed above. The T cells can be identified by producing synthetic versions of the peptides and bind them with commercially produced MHC or MHC-like proteins, then using these ex vivo complexes to bind to the target T cells. One should appreciate that the harvested T cells can included T cells that have been activated by the patient's immune response to the disease, exhausted T cells, or other T cells that are responsive to the discussed features.

Moreover, the immune competent cells may also be an avatar dendritic cell that mediates activation of NK cells and T-cells in contact/proximity to the tumor cell. For example, in one preferred aspect, the avatar dendritic cell is a chimeric molecule complex comprising (a) a fusion protein that includes an IL15 receptor portion, an Fc portion, and a first affinity portion, and (b) a fusion protein that includes an IL15 ligand portion, and a second affinity portion, wherein at least one of the first and second affinity portions bind to a neoepitope, a tumor specific antigen, or a tumor associated antigen. In especially preferred aspects, the avatar dendritic cell is based on an ALT-803 scaffold in which an IL-15-based immunostimulatory protein complex comprises two protein subunits of a human IL-15 variant associated with high affinity to a dimeric human IL-15 receptor a (IL-15Ra) sushi domain/human IgG1 Fc fusion protein (J Immunol (2009) 183: 3598-3607). The IL-15 variant is a 114 amino acid polypeptide comprising the mature human IL-15 cytokine sequence, with an asparagine to aspartate substitution at position 72 of helix C (N72D). The human IL-15Ra sushi domain/human IgG1 Fc fusion protein comprises the sushi domain of the human IL-15 receptor a subunit (IL-15Ra) (amino acids 1-65 of the mature human IL-15Ra protein) linked to the human IgG1 CH2-CH3 region containing the Fc domain (232 amino acids). Except for the N72D substitution, all of the protein sequences are human. In addition to the ALT-803 component, contemplated avatar dendritic cells include one or more targeting domains as is shown in a T×M scaffold (see URL: altorbioscience.com/our-science/i1-15-protein-superagonist-and-scaffold-technology/). Preferably, the targeting domains bind to a patient and tumor specific neoepitope or a tumor specific or tumor associated epitope. As a result, tumor cells are bound by the hybrid molecule on the basis of the neoepitope. The so bound hybrid molecule then provides via the IL15/IL15Ra portion a stimulatory signal to NK and T cells in the context of the neoepitope at the tumor cell and as such has a similar functional character as compared to an activated dendritic cell (hence the term avatar dendritic cell). Most typically, the targeting domain is a scFv with known binding specificity.

In still further contemplated aspects, it should be appreciated that the first and the second targeting domains may be the same (e.g., both domains will bind to a tumor and patient specific neoepitope) or different. Where the binding domains are different, it should be noted that the first binding domain will bind to a patient and tumor specific neoepitope or a tumor specific or tumor associated epitope while the second affinity portion that binds a mediator molecule that is involved in immune suppression. For example, suitable second affinity portions may bind specifically CD20, chromosome 16 open reading frame 82 (TNT), transforming growth factor 13 (TGFβ), IL-8, or may bind a checkpoint inhibitor ligand or receptor (e.g., bind to PD-L1, PD-L2, or CTLA4). Notably, such constructs operate in a manner similar to a dendritic cell with respect to target specific activation of T-cells and NK cells. Indeed, as the chimeric molecule has an IL15 portion (preferably a superagonist version) bound to the alpha chain of the IL-15 receptor, the so bound IL-15 strongly activates cells expressing the beta and gamma chain of the IL-15 receptor, which are found on T-cells and NK cells. Thus, using an avatar dendritic cell (particularly in combination with the targeting agent and cytokine or chemoattractant) will advantageously attract and activate NK cells and T-cells, stimulate their proliferation, and even lead to memory cell formation.

Alternatively, or additionally, tumor targeting engineered molecules may be employed to attract and activate various components of the innate and adaptive immune system. In one preferred aspect, the engineered molecule is an engineered antibody that is designed to reach the tumor microenvironment (TME) and that has multi-functional portions that interact with immune competent cells upon binding to tumor cells. For example, in one embodiment, the engineered antibody molecule disclosed herein may be a bispecific Fc-IgG1 fusion protein based on the avatar dendritic cell, where the protein molecule comprises an IgG1 Fc portion on one end (the “Fc end”), and where one former Fab end is replaced by a chemokine and/or a cytokine to attract various effector cells, while the other former Fab end is engineered to bind specifically to a tumor cell antigen.

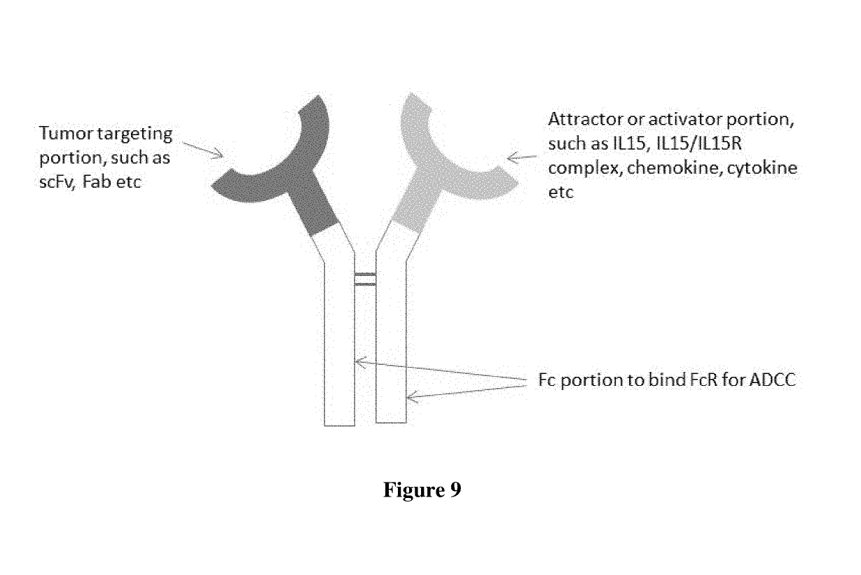

Such engineered hybrid molecules have several advantages. For example, in one embodiment, the Fc portion may act as a docking mechanism for NK cells, cytotoxic T cells, etc (that have an Fc receptor) to so mediate downstream signaling events such as antibody dependent cellular cytotoxicity (ADCC) and/or complement dependent cytotoxicity (CDC). In other words, the Fc end of the molecule is designed to attract and activate immune cells that mediate ADCC. Most typically, the IgG1 Fc portion of the hybrid molecule is engineered to bind to a corresponding Fc Receptor that can be found on macrophages, dendritic cells, and NK cells. Optionally or additionally, the IgG1 Fc portion may comprise an additional polypeptide chain to bind or attract further immune competent cells. This could include certain cytokines, chemokines, etc. Likewise, the IgG1 Fc portion may also comprise one or more additional polypeptides that act as a TGF-beta or IL-8 trap to so reduce signaling molecules that would otherwise activate to attract Tregs or MDSCs. It should be recognized that the Fc portion will not only serve as an activating ligand to cells having a Fc Receptor, but will also serve to facilitate transport of the hybrid molecule to the TME (e.g., via TcRn receptor on neovasculature in the TME). In addition, the Fc portion will also significantly extend serum half life time of the hybrid molecule and serve as an anchor point for two functional entities as is exemplarily and schematically illustrated in FIG. 9. Most typically, the hybrid molecule will be engineered to have two distinct arms.

With further reference to FIG. 9, the first of the two arms is an scFv portion or a Fab portion that has binding specificity to a tumor associated antigen, tumor specific antigen, or patient and tumor specific antigen to so enable tumor cell specific binding of the hybrid molecule to the tumor cell. The second of the two arms may then exhibit further immune activating function, preferably via chemokine activity to attract immune competent cells to the tumor cell and/or via cytokine activity to activate/enhance cytotoxicity of yet a further set of immune competent cells. Thus, it should be recognized that the hybrid molecules contemplated herein are specifically engineered to home to the tumor, attract, bind, and activate effector cells (e.g., innate NK, macrophages, monocytes), while another portion binds and activates NK and CD8+ cells via the Fc receptor. Notably, such hybrid molecules will not attract and/or activate Tregs and MDSCs. Alternatively, bi-specificity may also be achieved via several other constructs. One way is the construction of bispecific T-cell engagers (BiTEs), comprising two scFvs with specificities for CD3 and a target antigen expressed as one polypeptide chain. Another way is dual-affinity re-targeting molecules (DARTs) follow a similar principle, wherein a C-terminal disulfide bond covalently links two scFvs that are expressed as separate polypeptides to generate a more stable bispecific molecule. Another way is the use non-antibody-binding scaffolds, e.g. designed ankyrin repeat proteins (DARPins). DARPins are small, single-domain proteins derived from natural repeat proteins that can be engineered to bind diverse antigens.

Viewed form a different perspective, it should be appreciated that one arm of a bi-specific hybrid molecule is adapted to bind to or attract effector cells via e.g., IL-15, chemokines and cytokines, while the other arm of the bi-specific Fab comprises a ScFv or Fab, and is designed to specifically bind to an antigen on the tumor cell. Thus, in one sense, the engineered molecule acts as a matchmaker—making a match between adaptive and innate side of the immune system and the targeted tumor cell. Thus, in one aspect of the inventive subject matter, the hybrid molecules provide a multi-functional activating complex on the surface of a tumor cell, resulting in functional enhancement of antigen presenting cells and various effector cells.

The engineered molecules disclosed herein have the advantage of being specifically engineered to be able to enter the tumor microenvironment (typically via FcRn in the neovasculature), and once in the tumor microenvironment, to attract/bind/activate the effector cells of the immune system and the adaptive side (dendritic cells killer T cells) by using the IgG1 Fc side of the engineered molecule, while the other fab or IL-15 side has high affinity binding to NK cells and CD-8 cells, but not Treg cells.

Viewed from this perspective, the unique focus of the engineered hybrid molecule is to bring the killing cells to the killing fields rather than targeting the tumor as a direct therapeutic agent. In one way, the engineered molecule may be viewed as evolved Mabs which are designed to target tumors. Until now, checkpoint Mabs were designed for T cells, but the advantage of the presently disclosed engineered hybrid molecule is that it is designed to accommodate all killing cells of the immune system, while also targeting the endothelial cell of the vasculature and optionally lymphatics. Thus, the engineered molecule is targeting multiple entities such as endothelial cells, NK cells, dendritic cells, T cells, monocytes etc.

Moreover, it should be appreciated that the IL15/IL15Ra portion also exerts inhibitory effect on immune suppressor cells, and particularly on Tregs and MDSCs. Viewed from another perspective, contemplated methods as described herein will promote formation of activated and proliferating NK and cytotoxic T-cells, memory NK cells expressing NKG2C, memory T-cells, and T-cells that act like NK cells via their NKG2D properties.

In addition to cell-based therapy or as an alternative, immune therapy may be performed by administration of a cancer vaccine composition, and especially a vaccine composition that uses one or more cancer neoepitopes that are specific to the cancer and the patient, or that uses cancer associated (CEA, MUC1, brachyury, etc.) or cancer specific (PSM, PSMA, HER2, etc.) antigens. As will be readily appreciated, such vaccine compositions may be delivered as viral vaccine (e.g., via recombinant adenovirus) that infects a patient's dendritic cells and/or as bacterial or yeast vaccine that is processed by dendritic cells of the patient.

Consequently, it should be recognized that the access to the tumor microenvironment and the delivery of an affinity agent with a chemoattractant to the tumor microenvironment will generate a constellation in the tumor microenvironment that enhances a directed cellular response to the tumor. Therefore, the inventor also contemplates a method of treating a patient diagnosed with a tumor, comprising: administering to a tumor microenvironment a chimeric molecule complex comprising (a) a fusion protein that includes an IL15 receptor portion, an Fc portion, and a first affinity portion, and (b) a fusion protein that includes an IL15 ligand portion, and a second affinity portion; wherein at least one of the first and second affinity portions bind to a neoepitope, a tumor specific antigen, or a tumor associated antigen; and administering to the tumor microenvironment an inhibitor of immune suppressor cells.

In addition, it is contemplated that the tumor microenvironment may be further exposed to a compound or composition that reduces presence, recruitment, activity, and/or proliferation of immune suppressor cells, and especially to one or more pharmaceutical agents that reduce activity and/or proliferation of Tregs and MDSCs. Therefore, particularly suitable agents include cisplatin, gemcitabine, 5-fluorouracil, cyclophosphamide, doxorubicin, temozolomide, docetaxel, paclitaxel, trabectedin, and RP-182 (see e.g., U.S. Pat. No. 9,492,499). Alternatively or additionally, administration of IMiDs (immunomodulatory drugs) and histone deacetylating drugs (HDAC) is contemplated to further reduce presence, recruitment, activity, and/or proliferation of immune suppressor cells, including Tregs and MDSCs. Such drugs will typically be administered using conventional dosages and treatment regiments. In further contemplated aspects, inhibition of suppressor cells may also be done using albumin bound drugs (e.g., nab-paclitaxel) during breaching the of the tumor microenvironment.

Therefore, the inventor also contemplates a method of treating a patient diagnosed with a tumor that includes a step of killing cells within a tumor microenvironment, and delivering a targeting agent to the killed cells in the tumor microenvironment wherein the targeting agent further comprises a signaling component. The signaling component is then used to attract a plurality of immune competent cells, and in a further step an inhibitor of immune suppressor cells is administered to the tumor microenvironment.

In one exemplary aspect of the inventive subject matter, the tumor microenvironment can be breached by administration of Bevacizumab (e.g., 5 mg/kg IV) and nanoparticulate albumin to which paclitaxel is coupled (Abraxane (Nab-paclitaxel) (e.g., 100 mg IV). Advantageously paclitaxel will also contribute to cell killing. Such treatment can be given, for example, over two to four weeks and may overlap tumor cell killing. For example, tumor cell killing can be done during and after breach of the tumor microenvironment with cisplatin (e.g., 40 mg/m² IV) and repeated stereotactic body radiation therapy (e.g., not to exceed 8 Gy). Overlapping or concomitant necrosis targeting may be achieved using an anti-neoepitope T×M (e.g., 10 μg/kg, s.c.), which is preferably given to the patient between 10-120 minutes prior to cell based therapy. In further contemplated aspects, the cell based therapy comprises an infusion with aNK or haNK cells (e.g., 2×10⁹ cells/dose IV). Furthermore, during the entire course of treatment, or after cell killing, or after necrosis targeting, suppressor cells may be inhibited by administration of various drugs, and especially administration of cyclophosphamide (e.g., 50 mg PO twice a day) and/or 5-FU (e.g., 400 mg/m² continuous IV infusion over 24 hours).

Of course, it should be recognized that the particular drug(s), dosages, and schedules will vary and will at least in part be dictated by the type of tumor, severity of disease, and patient history. Therefore, numerous other treatment modalities are also deemed appropriate. For example, suitable inhibitors for suppressor cells include cisplatin, gemcitabine, 5-fluorouracil, capecitabine, cyclophosphamide, doxorubicin, temozolomide, docetaxel, paclitaxel, trabectedin, and RP-182. As will be appreciated, such compounds may be coupled to albumin (preferably nanoparticulate albumin) to take advantage of gp60-specific mediated entry into the tumor microenvironment, or to a pH sensitive carrier gel (see e.g., Nano Lett. 2017 Oct. 11; 17(10):6366-6375). Therefore, it should be recognized that breaching the tumor microenvironment and inhibiting suppressor cells may be performed in a combined manner Additionally, it is contemplated that the inhibition of immune suppression can also be done using one or more checkpoint inhibitors, such as avelumab and ipilimumab.

Likewise, it should be appreciated that the cell based therapy need not be limited to use of haNK cells, but that the cell based therapy may be using aNK cells, taNK, CAR-T cells, etc. Moreover, it is contemplated that the cell based therapy may also use transfusion of the patient's own dendritic cells (which may have been exposed to a vaccine composition or neoepitopes of the patient) or T cells. Where T cells are used, it is particularly preferred that such T cells include reactivated anergic T cells or genetically engineered T-cells.

Moreover, it is contemplated that the call based therapy may be assisted by vaccine compositions, especially where the cell based therapy is based on the patient's own immune competent cells (which may be already present in the patient and thus not require any transfusion. For example, suitable vaccine compositions include adenoviral vaccine compositions such as ETBX-021: ETBX-021 is a HER2-targeting adenovirus vector vaccine comprising the Ad5 [E1−, E2b−] vector and a modified HER2 gene insert (Cancer gene therapy 2011; 18:326-335). The HER2 gene insert encodes a truncated human HER2 protein that comprises the extracellular domain and transmembrane regions. The entire intracellular domain, containing the kinase domain that leads to oncogenic activity, is removed; or ETBX-051 (Ad5 [E1−, E2b−]-Brachyury): ETBX-051 is an Ad5-based adenovirus vector vaccine that has been modified by the removal of the E1, E2b, and E3 gene regions and the insertion of a modified human Brachyury gene. The modified Brachyury gene contains agonist epitopes designed to increase cytotoxic T lymphocyte (CTL) antitumor immune responses (see e.g., Oncotarget. 2015; 6:31344-59); ETBX-061 (Ad5 [E1−, E2b−]-MUC1): ETBX-061 is an Ad5-based adenovirus vector vaccine that has been modified by the removal of the E1, E2b, and E3 gene regions and the insertion of a modified human MUC1 gene. The modified MUC1 gene contains agonist epitopes designed to increase CTL antitumor immune responses (see e.g., Oncotarget. 2015; 6:31344-59).

Yeast based vaccines may also be employed and exemplary yeast based vaccine compositions include GI-4000 (GI-4014, GI-4015, GI-4016, GI-4020): GI-4000 is 4 separate products from the GI-4000 series, GI-4014, GI-4015, GI-4016, GI-4020. Each of these is a recombinant, heat-inactivated S. cerevisiae engineered to express a combination of 2-3 of the 6 mutated Ras oncoproteins. GI-4014, GI-4015, and GI-4016 products each contain two mutations at codon 61 (glutamine to arginine [Q61R], and glutamine to leucine [Q61L], plus one of three different mutations at codon 12 (either glycine to valine [G12V], glycine to cysteine [G12C], or glycine to aspartate [G12D]). GI-4020 product contains two mutations at codon 61 (glutamine to histidine R61141 and glutamine to leucine [Q61L]), plus one mutation at codon 12 (glycine to arginine [G12R]). Thus, GI-4000 is manufactured as four individual products with the subnames GI-4014, GI-4015, GI-4016, and GI-4020 depending on the mutated Ras oncoprotein the product is engineered to express. The biologic product is formulated in phosphate buffered saline (PBS) for injection and vialed separately at a concentration of 20YU/mL (1YU=10⁷ yeast cells). Each single use 2 mL vial contains 1.2 mL of biologic product. Two vials of drug product will be used for each GI-4000 administration visit. The specific GI-4000 product containing the Ras mutation in the subject's tumor will be used for treatment (GI-4014 for G12V, GI-4015 for G12C, GI-4016 for G12D, GI-4020 for G12R or Q61H, and GI-4014, GI-4015, or GI-4016 for Q61L or Q61R). Two syringes of 0.5 mL will be drawn from each vial, and 4 total injections will be administered for a dose of 40YU at each dosing visit.

GI-6207: GI-6207 is a heat-killed, recombinant Saccharomyces cerevisiae yeast-based vaccine engineered to express the full length human carcinoembryonic antigen (CEA), with a modified gene coding sequence to code for a single amino acid substitution (asparagine to aspartic acid) at the native protein amino acid position 610, which is designed to enhance immunogenicity. A plasmid vector containing the modified human CEA gene is used to transfect the parental yeast strain (S. cerevisiae W303—a haploid strain with known mutations from wild-type yeast) to produce the final recombinant vaccine product (see e.g., Nat Med. 2001; 7:625-9); GI-6301: GI-6301 is a heat-killed, S. cerevisiae yeast-based vaccine expressing the human Brachyury (hBrachyury) oncoprotein. The Brachyury antigen is the full-length protein possessing an N-terminal MADEAP (Met-Ala-Asp-Glu-Ala-Pro) motif appended to the hBrachyury sequence to promote antigen accumulation within the vector and a C-terminal hexahistidine epitope tag for analysis by Western blotting (see e.g., Cancer Immunol Res. 2015; 3:1248-56). Expression of the hBrachyury protein is controlled by a copper-inducible CUP1 promoter.

With respect to suitable avatar dendritic cells it should be noted that avatar dendritic cells may have distinct targeting domains that can be specific to the patient tumor's specific neoepitopes, and/or specific to one or more tumor associated or tumor specific antigens. In addition, and as noted before, the avatar dendritic cell may also have a targeting domain that is used to deplete the tumor microenvironment of one or more immune suppressive factors, and especially of IL-8 and/or TGF-beta to so allow for enhanced immune stimulation in the context of tumor antigens.

THE NANT CANCER VACCINE: In view of the above contemplations and examples, it should be recognized that The NANT Cancer Vaccine is a modern approach and paradigm change to current traditional regimens of cancer therapy—a regenerative advanced therapy to maximize immunogenic cell death (ICD) while maintaining and augmenting the patients' antitumor adaptive and innate responses to cancers. The NANT Cancer Vaccine therapy makes use of lower, metronomic doses of both cytotoxic chemotherapy and radiation therapy, with the aim of inducing damage associated molecular pattern (DAMP) signals and tumor cell death while minimizing suppression of the immune system. These treatments are combined with immunomodulatory agents, checkpoint inhibitors, and fusion proteins that serve to augment and stimulate patients' adaptive and innate immune responses. By overcoming the immunosuppressed (escape) tumor microenvironment, the elimination phase of cancer can be reinstated through effector cells (mature dendritic cells, NK cells, cytotoxic T-cells, memory T-NK cells), activated by the NANT Cancer Vaccine combination therapy of fusion proteins, adenovirus and yeast vector vaccines, and natural killer cells.

The NANT Cancer Vaccine is administered in a spatiotemporal delivery of combination immunotherapeutic products to immunomodulate the tumor microenvironment, activate the innate adaptive immune system and to induce immunogenic cell death (ICD). The inventor hypothesized, that by inducing immunogenic cell death and protecting the innate and adaptive immune system, the NANT Cancer Vaccine will result in long term sustainable remission of multiple tumor types with lower toxicity and higher efficacy than current standards of care. In one contemplated example, the vaccine is administered through the following sequential elements over a cycle of 14-days to:
a. Break the Escape Phase of Cancer Immunoediting:
   Overcoming the tumor immunosuppressed state, informed by tissue and liquid biopsies, with low-dose metronomic chemotherapeutic agents capable of inhibiting T-Reg, MDSC's, and M2 Macrophages
   Inhibiting cytokines (TGF (3) which enhance immunosuppressive immune system
b. Induce the Elimination Phase of Cancer Immunoediting:
   Upregulating induction of damaged associated molecular pattern (DAMP) signals, upregulate tumor associated MHC restricted antigens and NK stress receptors ligands (NKG2D ligands), upregulate tumor specific receptor ligands such as PD-L1 through low-dose radiation, immunomodulatory drugs (IMiDs) and histone deacetylase (HDAC) agents.
   Activating dendritic cells, natural killer cells, cytotoxic T-cells, memory T & Natural Killer (NK) cells through adenovirus & yeast vector vaccines, cytokine fusion protein administration, checkpoint inhibitors and NK cell therapy infusion.
c. Reinstate the Equilibrium Phase of Cancer Immunoediting:
   Maintaining TH1 status with vaccine boosters, cytokine fusion protein maintenance and or regular exogenous NK infusions.

The spatiotemporal administration of the NANT Cancer Vaccine product has the potential to reinstate the natural state of the patient's immune system by overcoming the escape phase, reestablishing the elimination phase and accomplishing long term maintenance by supporting the equilibrium phase of immunoediting.

Key Biological Elements of the NANT Cancer Vaccine Product: It is generally contemplated that these elements are administered in combination to activate the innate and adaptive immune system to induce immunogenic cell death are:
a. N: Nab—Nanoparticle Albumin Bound (Nab) chemotherapy combinations to enter the tumor microenvironment (transcytosis) to overcome the tumor suppressor environment—the human protein component.
b. A: Antigen—Adenoviral & Yeast vectors delivering tumor associated and neoantigens to activate immature Dendritic Cells (DC)—the molecularly engineered tumor associated & neoantigen component.
c. N: Natural Killer—Activating endogenous Natural Killer (NK) cells via cytokine administration (IL-15, IL-12, IL-18) and infusing genetically modified Natural Killer cell line (NK-92)—the endogenous and exogenous natural killer cell component.
d. T: T-Cells—Sustaining long term remission by memory T-cell & NK cells through vaccine, cell therapy and fusion protein maintenance—the genetically engineered fusion protein cytokine stimulator and checkpoint inhibitor component

FIG. 5 exemplarily illustrates such approach addressing the three phases of immunoediting. The intent of the NANT Cancer Vaccine development effort is to employ this novel treatment protocol in a series of clinical trials in which the therapy will be investigated across multiple oncology indications. The first NANT Cancer Vaccine clinical trial will be in pancreatic cancer under Protocol QUILT 3.039, titled “NANT Pancreatic Cancer Vaccine: Combination Immunotherapy in Subjects with Pancreatic Cancer who have Progressed on or after Standard-of-Care Therapy”. Examples of the specific products which accomplish overcoming the suppressive tumor environment, inducing the elimination phase with adenoviruses, tumor associated antigens and natural killer cell platform are provided below. Small variations in the chemotherapies and their doses will be based upon past experiences with these therapies in a given indication. Specific protocols will be designed to accommodate these products and minor variations specific to the indication.

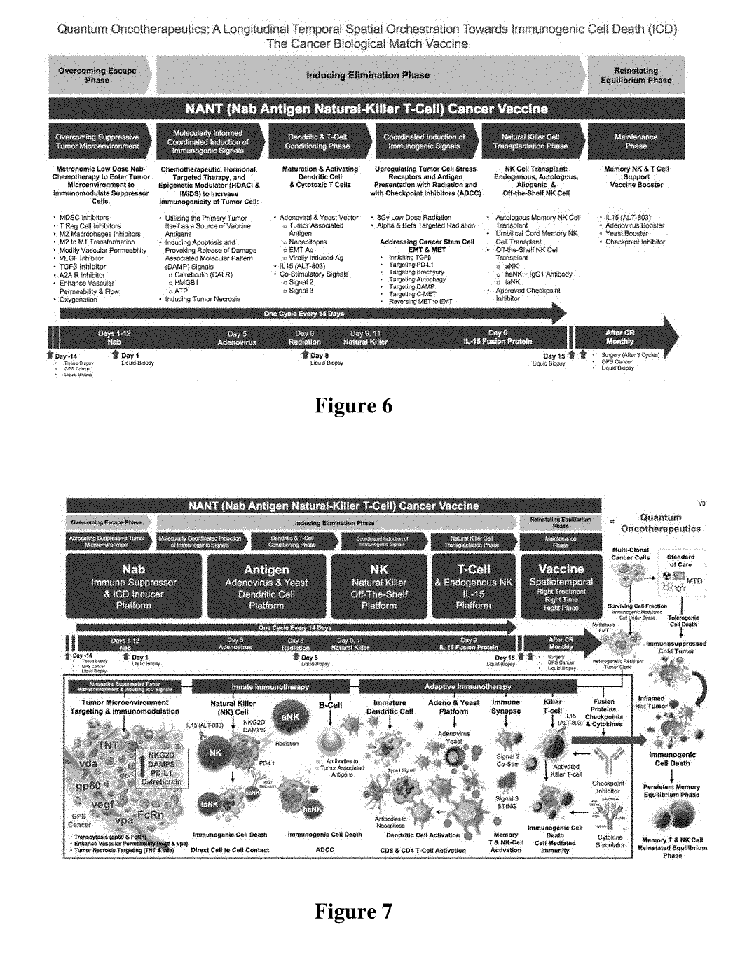

Similarly, FIG. 6 exemplarily illustrates the NANT cancer vaccine key biological elements administered over 14-day cycle. Mechanistically, the spatiotemporal delivery of combination immunotherapeutic products (The NANT Cancer Vaccine) will immunomodulate the tumor microenvironment, induce immunogenic cell death (ICD) and result in long term sustainable remission of multiple tumor types with lower toxicity and higher efficacy than current standards of care by:
a. Penetrating the tumor microenvironment to overcoming the tumor immunosuppressed state, informed by tissue and liquid biopsies, with low-dose metronomic chemotherapeutic agents capable of inducing immunogenic cell death (ICD) with inhibitors of immunosuppressive cytokines.
b. Upregulating induction of damaged associated molecular pattern (DAMP) signals, upregulate tumor associated MHC restricted antigens and stress receptors (NKG2D) through low-dose radiation, IMiDs and HDAC agents
c. Activating dendritic cells, natural killer cells, cytotoxic T-cells, memory T & NK cells through cytokine fusion protein, checkpoint inhibitor administration and NK cell therapy infusion.
d. Maintaining the equilibrium state through vaccine, NK and fusion protein boosters

The inventor hypothesizes that this combination product of cell therapy, biological proteins, and genetically engineered vaccines (NANT cancer vaccine) will induce immunogenic cell death and result in durable responses across multiple tumor types with lower toxicity than the traditional treatment regimens administered as the current standards of care, as is exemplarily shown in FIG. 7.

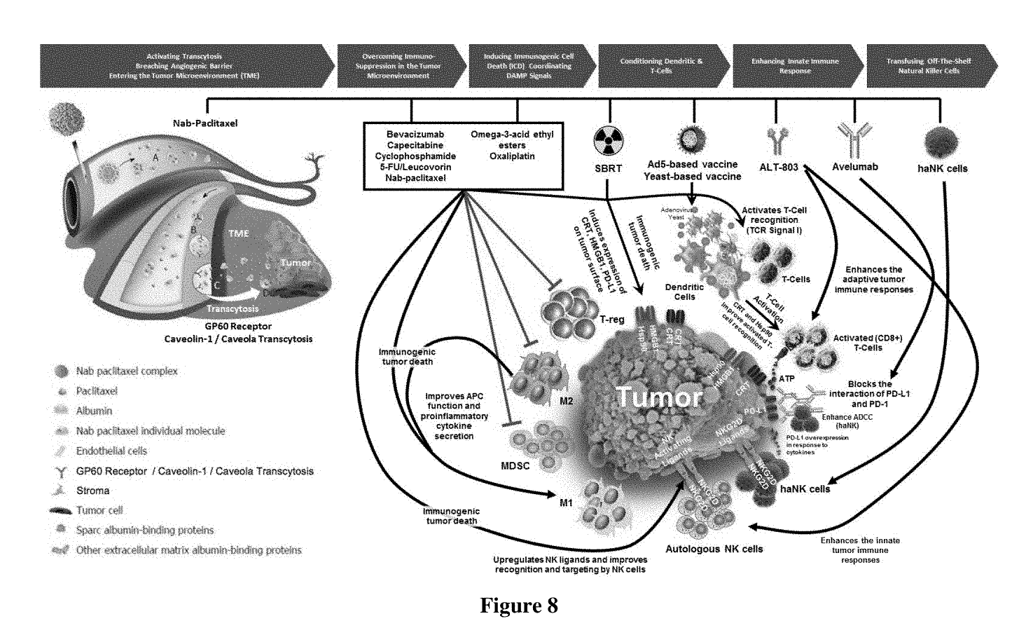

FIG. 8 exemplarily illustrates a treatment regimen and associated effects by the treatment modalities as presented herein. Of course, it should be appreciated that instead of (or in addition to) use of ALT-803 one or more avatar dendritic cells as described herein may be employed. For example, a particularly preferred avatar dendritic cell may comprise a T×M based molecule that has targeting moieties that specifically bind to patient and tumor specific neoepitopes. Such avatar dendritic cell may be administered during or after induction of immunogenic cell death and/or radiation therapy. Likewise, it should be appreciated that the targeting agent that is administered to the killed cells in the tumor microenvironment may be given to the patient during or after induction of immunogenic cell death and/or radiation therapy. Particularly suitable targeting agents will include those that target tumor necrosis proteins (e.g., calreticulin, Hsp90, histone proteins (e.g., HMGB1) and that include one or more chemokines (e.g., CXCL14) as a chemoattractant.

Moreover, it should be appreciated that complementary diagnostics to the NANT cancer vaccine may be employed, and especially GPS Cancer (whole genome sequencing, transcriptome sequencing, tumor vs. matched normal mutational analysis, quantitative proteomics) and liquid ctDNA and/or ctRNA Biopsies. Thus, throughout the course of the NANT Cancer Vaccine administration, comprehensive genomic, transcriptomic, and proteomic profiling (Omics Analysis) of the patient's tumor and blood will be used to inform and follow the spatiotemporal longitudinal tumor status and to provide a precise picture of the ongoing evolution of the tumor. This complementary diagnostic tissue and liquid biopsy analysis will enable precision therapy (surgery, chemotherapy, radiotherapy and immunotherapy) based on the unique molecular signature of the tumor across time and space, independent of anatomy (Quantum Oncotherapeutics) to achieve the optimal therapeutic outcome.

Further contemplated compounds, compositions, aspects, and examples suitable for use herein are disclosed in our co-pending International application with the serial number PCT/US17/40297, incorporated by reference herein.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Where the specification claims refers to at least one of something selected from the group consisting of A, B, C . . . and N, the text should be interpreted as requiring only one element from the group, not A plus N, or B plus N, etc.

What is claimed is:

1. A method of treating a patient diagnosed with a tumor, comprising:
breaching a vasculature feeding the tumor to thereby increase delivery of at least one of a drug and an immune competent cell into a tumor microenvironment;
killing cells within the tumor microenvironment;
delivering a targeting agent to the killed cells in the tumor microenvironment wherein the targeting agent further comprises a signaling component; and
providing to the tumor microenvironment (a) a cell-based therapy or avatar dendritic cell or a multi-functional hybrid molecule, and (b) an inhibitor of immune suppressor cells.

2. The method of claim 1 wherein the step of breaching the vasculature comprises a step of targeting at least one of a gp60 transporter and a neonatal Fc receptor (FcRn).

3. The method of claim 2 wherein targeting the gp60 transporter comprises contacting the gp60 transporter with a drug coupled to an albumin nanoparticle.

4. The method of claim 2 wherein targeting the FcRn comprises contacting the FcRn with a drug coupled to an Fc portion of an IgG.

5. The method of claim 3 or claim 4 wherein the drug is a cytotoxic drug, a vascular disrupting agent, or a cytokine.

6. The method of claim 1 wherein the step of breaching the vasculature comprises a step of contacting the vasculature with at least one of NO, IL-2, a VEGF receptor inhibitor, and a permeability enhancing peptide (PEP), and optionally wherein contacting the vasculature with the at least one of the NO, the IL-2, the VEGF receptor inhibitor, and the PEP is performed locally.

7. The method of claim 1 wherein the killing cells within the tumor microenvironment is performed using at least one of radiation, low-dose chemotherapy, a drug coupled to an albumin nanoparticle, and a drug coupled to an Fc portion of an IgG.

8. The method of claim 1 wherein the targeting agent comprises an affinity agent that binds to at least one of a nucleolin, DNA, and a histone.

9. The method of claim 8 wherein the affinity agent comprises an antibody or fragment thereof.

10. The method of claim 1 wherein the signaling component comprises a chemoattractant or an immune stimulatory cytokine.

11. The method of claim 10 wherein the chemoattractant comprises a chemokine that attracts at least one of a T-cell, an NK cell, a dendritic cell, and a macrophage, or wherein the immune stimulatory cytokine comprises IL-2, IL-15, a modified IL-15, or IL-21.

12. The method of claim 1 wherein the cell-based therapy comprises a dendritic cell, an activated dendritic cell, a dendritic cell infected with a virus that contains a nucleic acid encoding at least one of a neoepitope, a cancer associated antigen, and a cancer specific antigen, an avatar dendritic cell, an autologous NK cell, an activated NK cell (aNK), a high-affinity NK cell (haNK), a target activated NK cell, a T-cell, and/or a CAR T-cell.

13. The method of claim 1 wherein the inhibitor of the immune suppressor cells comprises an inhibitory peptide for a mannose receptor, 5-FU, a phosphodiesterase-5 inhibitor, a COX-2 inhibitor, or cyclophosphamide, and optionally wherein the inhibitor of the immune suppressor cells is bound to albumin.

14. The method of claim 1 further comprising administering IL-15 or a IL-15 superagonist to the patient.

15. A method of treating a patient diagnosed with a tumor, comprising:
administering to a tumor microenvironment a chimeric molecule complex comprising (a) a fusion protein that has an IL15 receptor portion, an Fc portion, and a first affinity portion, and (b) a fusion protein that has an IL15 ligand portion, and a second affinity portion;
wherein at least one of the first and second affinity portions bind to a neoepitope, a tumor specific antigen, or a tumor associated antigen; and
administering to the tumor microenvironment an inhibitor of immune suppressor cells.

16. The method of claim 15 further comprising administering to the patient an autologous NK cell, an activated NK cell (aNK), a high-affinity NK cell (haNK), a target activated NK cell, and/or a T-cell.

17. The method of claim 15 wherein the step of administering to the tumor microenvironment is performed across the vasculature of the tumor microenvironment and/or comprises a step of increasing permeability of the vasculature of the tumor microenvironment.

18. The method of claim 15 further comprising a step of treating the tumor microenvironment with a targeting agent comprising a signaling component and an affinity agent that binds to at least one of a nucleolin, DNA, and a histone.

19. The method of claim 18 wherein the signaling component comprises a chemokine or an immune stimulatory cytokine.

20. The method of claim 15 further comprising a step of killing cells within the tumor microenvironment.

21. A method of treating a patient diagnosed with a tumor, comprising:
killing cells within a tumor microenvironment, and delivering a targeting agent to the killed cells in the tumor microenvironment wherein the targeting agent further comprises a signaling component;
using the signaling component to attract a plurality of immune competent cells; and
administering to the tumor microenvironment an inhibitor of immune suppressor cells.

22. The method of claim 21 wherein killing cells within the tumor microenvironment is performed using at least one of radiation, low-dose chemotherapy, a drug coupled to an albumin nanoparticle, and a drug coupled to an Fc portion of an IgG.

23. The method of claim 21 wherein the targeting agent comprises an affinity agent that binds to at least one of a nucleolin, DNA, and a histone.

24. The method of claim 21 wherein the signaling component comprises a chemoattractant.

25. The method of claim 24 wherein the chemoattractant comprises a chemokine that attracts at least one of a T-cell, an NK cell, a dendritic cell, and a macrophage.

26. The method of claim 21 wherein the immune competent cells comprise autologous NK cells, activated NK cells (aNK), high-affinity NK cells (haNK), target activated NK cells, T-cells, T-cells expressing a chimeric antigen receptor, and/or dendritic cells expressing at least one of a neoepitope, a cancer associated antigen, and a cancer specific antigen.

27. The method of claim 21 wherein the immune competent cells are administered to the patient after the step of delivering the targeting agent to the patient.

28. The method of claim 21 wherein the immune competent cells are genetically engineered NK cells or dendritic cells expressing a recombinant gene.

29. The method of claim 21 further comprising a step of increasing permeability of vasculature feeding the tumor microenvironment.

30. The method of claim 29 wherein increasing permeability of the vasculature comprises a step of contacting the vasculature with at least one of NO, IL-2, a VEGF receptor inhibitor, and a permeability enhancing peptide (PEP).

31. A method of treating a patient diagnosed with a tumor, comprising:
administering to a tumor microenvironment a hybrid protein comprising an Fc portion and a first and a second binding portion;
wherein the Fc portion is adapted to bind to a Fc receptor on a macrophage, dendritic cell, or NK cell;
wherein the first binding portion comprises a tumor targeting motif, and wherein the second binding portion comprises a cytokine or a chemokine portion; and
administering to the tumor microenvironment an inhibitor of immune suppressor cells.

32. The method of claim 31 further comprising administering to the patient an autologous NK cell, an activated NK cell (aNK), a high-affinity NK cell (haNK), a target activated NK cell, and/or a T-cell.

33. The method of claim 31 wherein the step of administering to the tumor microenvironment is performed across the vasculature of the tumor microenvironment and/or comprises a step of increasing permeability of the vasculature of the tumor microenvironment.

34. The method of claim 31 further comprising a step of treating the tumor microenvironment with a targeting agent comprising a signaling component and an affinity agent that binds to at least one of a nucleolin, DNA, and a histone.
